(19) United States
(12) Patent Application Publication
(10) Pub. No.: US 2021/0008299 A1
(43) Pub. Date: Jan. 14, 2021

(54) EPIDERMAL SUSPENSION SPRAY DEVICE

(71) Applicant: College of Engineering, Pune, Pune (IN)

(72) Inventors: Saiprasad Poyarekar, Pune (IN); Chetan Mahale, Pune (IN); Sandip Anasane, Pune (IN); Bharatkumar Ahuja, Pune (IN); Nikhil Panse, Pune (IN); Rohit Phulwar, Pune (IN)

(21) Appl. No.: 17/034,850

(22) Filed: Sep. 28, 2020

Related U.S. Application Data
(63) Continuation of application No. PCT/IN2019/050250, filed on Mar. 28, 2019.

(30) Foreign Application Priority Data
Mar. 28, 2018 (IN) 201821011504

(51) Int. Cl. A61M 11/02; A61M 15/00

(52) U.S. Cl. CPC A61M 11/02; A61M 15/0006; A61M 15/0028; A61M 2202/09; A61M 15/002; A61M 2205/071; A61M 2205/8225; A61M 15/0013

(57) ABSTRACT

A dermo-epidermal suspension spray device, said device comprising: a refill capsule (H) to contain skin pieces, said refill capsule (H) placed inside a sample loader (B); a stirrer mechanism to be connected to said refill capsule (H/14/24) so that a solution of skin pieces and saline is continuously stirred to maintain a uniformly suspended solution or skin pieces and saline in a non-homogenous stirred manner; a nozzle (A) to spray said uniformly suspended solution from said refill capsule (H/14/24) onto a portion of a body where grafting is required; an actuator (D) to enable spraying of said uniformly suspended solution; and air container (G) to store compressed air, said air container (G) connected to said refill capsule (H) so that said compressed air is used for spraying said uniformly suspended solution of skin pieces and saline through said nozzle (A).

### RELATED APPLICATIONS

This application claims priority under 35 U.S.C. § 120 to, and is a continuation of, co-pending International Application PCT/IN2019/050250, filed Mar. 28, 2019 and designating the US, which claims priority to IN Application 201821011504, filed Mar. 28, 2018, such IN Application also being claimed priority to under 35 U.S.C. § 119. These IN and International applications are incorporated by reference herein in their entireties.

### BACKGROUND

### Field

This invention relates to the field of biomedical engineering. Particularly, this invention relates to an epidermal suspension spray device.

Skin grafting is a type of graft surgery involving the transplantation of skin. The transplanted tissue is called a skin graft.

Skin grafting is often used to treat:
Extensive wounding or trauma
Burns

Areas of extensive skin loss due to infection such as necrotizing fasciitis or purpura fulmina.

Specific surgeries that may require skin grafts for healing to occur are most commonly removal of skin cancers.

Generally, skin grafting is done for burn patients where the percentage of involvement is less, and the surgeon requires an area from where skin can be harvested and used as a skin graft. But, in patients, where there are major burns, and there is limited area from where the surgeon can take a skin graft, there is a problem. In such cases, use a very small area of graft can be used to cover a large surface area.

Skin grafting is routinely performed for the management of wounds due to trauma or burns. But, in patients where the raw areas because of burns or trauma are extensive and the area to harvest the skin graft from the patient is very limited pose a lot of difficulty. In such cases, the skin graft is meshed to a variable degree to permit its expansion. However, in many cases, even expansion might not be sufficient to meet the requirements of the skin.

A dermo-epidermal spray or a skin spray may be required in such instances. If such a spray is not used in a controlled manner, it results in uneven skin distribution and recurrent blockage of the spray.

### SUMMARY

An object of the invention is to provide a device which provides to spray minced skin pieces in controlled manner.

Another object of the invention is to provide a device which provides to spray minced skin pieces in controlled manner to cover a large area of wound, thereby aiding faster recovery.

According to this invention, there is provided a dermo-epidermal suspension spray device, said device comprising:
a refill capsule configured to contain skin pieces, said refill capsule being placed inside a sample loader;
a stirrer mechanism configured to be connected to said refill capsule so that a solution of skin pieces and saline is continuously stirred to maintain a uniformly suspended solution or skin pieces and saline in a non-homogenous stirred manner;
a nozzle attachment in order to spray said uniformly suspended solution from said refill capsule onto a portion of a body where grafting is required;
an actuator in order to enable spraying of said uniformly suspended solution on to said portion of body; and
air container configured to store compressed air, said air container connected to said refill capsule so that said compressed air is used for spraying said uniformly suspended solution of skin pieces and saline through said nozzle attachment.

In at least an embodiment, said stirrer mechanism is a motorized shaker which is the electronic stirrer mechanism.

In at least an embodiment, said stirrer mechanism is a motorized shaker comprising:
a motor with cam shaft which provides vibrations and, therefore, stirring action to the solution of skin pieces along with saline in said refill capsule; and
said container located on a resilient means to allow for free stirring action of said refill capsule.

In at least an embodiment, said stirrer mechanism is a magnetic shaker comprising:
a refill capsule with a solution of skin pieces along with saline, which solution is to be shaken/stirred; and
an outer, angularly displaceable, magnetic disc driven by a motor which cooperates magnetically with an inner magnet located inside said refill capsule to cause said refill capsule to angularly displace when the refill capsule so as to shake/stir the contents within.

In at least an embodiment, said device comprises a delivery pipe to feed said uniformly suspended solution to said nozzle, characterised in that, said delivery pipe follows golden ration of length vis-a-vis diameter of pipe to ensure effective spray from said nozzle.

In at least an embodiment, said device comprises main connector in order to detach sample loaded (B) from said device body for refilling said capsule, after use.

In at least an embodiment, said air container comprises a pressure gauge and allows a user to determine pressure of spray.

In at least an embodiment, said air container being coupled with a hand pump attached for manual filling of air inside said air container.

Typically, said device comprises electronically controlled valves and stirrer, in said stirrer mechanism, provided to provide specificity and sensitivity to said device.

Preferably, said device comprises a flow control valve, controlled by a stepper motor, provided to control spray through said nozzle, said stepper motor being used to vary opening of flow control valve to control air flow emerging from said device.

### BRIEF DESCRIPTION OF THE DRAWINGS

The invention will now be described in relation to the accompanying drawings, in which:

FIG. 1 illustrates a pictorial representation of an autologous skin graft;

FIG. 2 illustrates an isometric views of the spray device;

FIG. 3 illustrates an exploded view of the spray device;

FIG. 4 illustrates an electronic stirrer mechanism of the spray device;

FIG. 5 illustrates a magnetic stirrer mechanism of the spray device;

FIG. 6 illustrates a perspective view of the spray device of FIG. 2;

FIG. 7 illustrates a flowchart of working of this spray device; and

FIG. 8 illustrates an experimental set-up to test operational parameters of the device of this invention.

### DETAILED DESCRIPTION

According to this invention, there is provided a dermo-epidermal suspension spray device

A small strip of skin is harvested, and minced into very small pieces. Then a suspension is made with help of normal saline. This is sprayed over the burn wound. In other words, these tiny pieces of skin act as seeds from where the skin generates, and over a due course of time, the wound is covered.

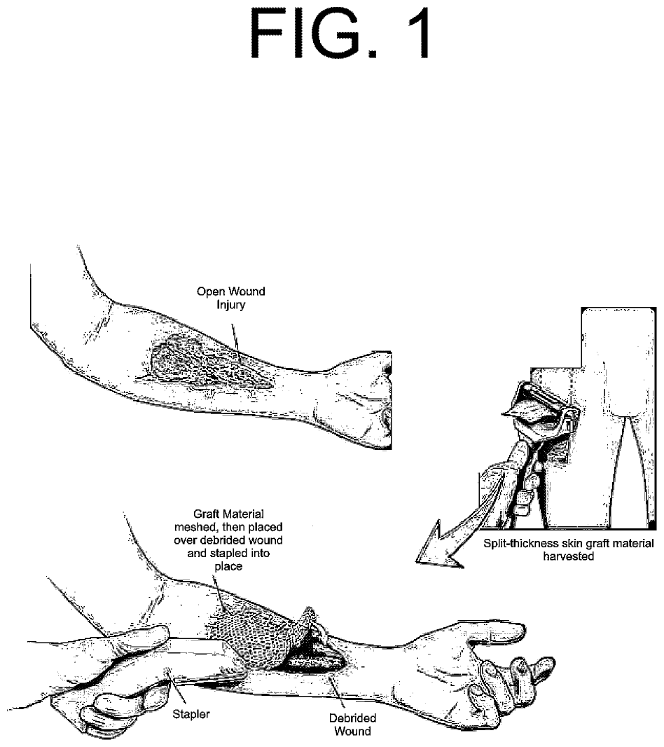

FIG. 1 illustrates a pictorial representation of an autologous skin graft.

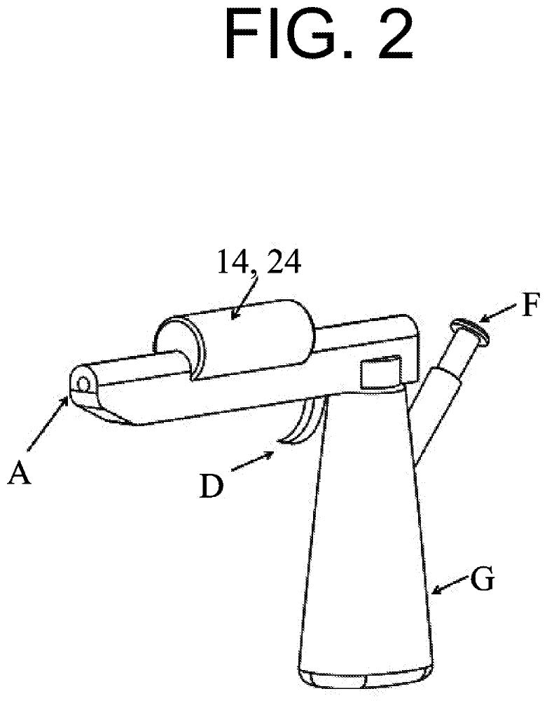

FIG. 2 illustrates an isometric view of one embodiment spray device.

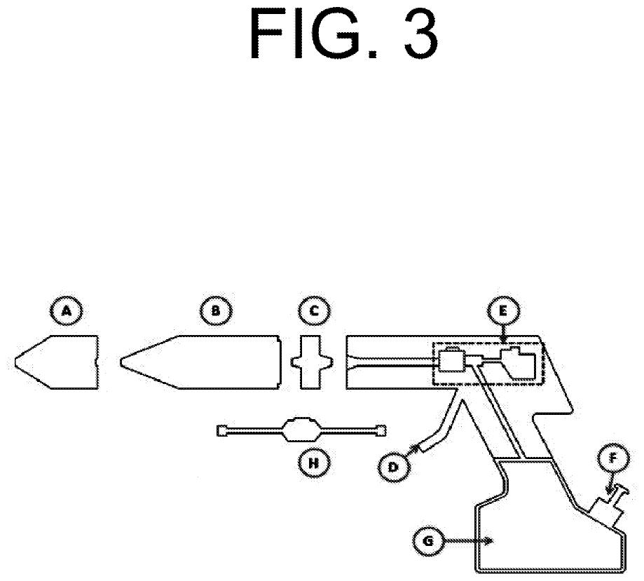

FIG. 3 illustrates an exploded view of the embodiment of spray device of FIG. 2.

In at least an embodiment, the spray device comprises a refill capsule (H) configured to contain skin pieces. The refill capsule (H) is placed inside a sample loader (B).

In at least an embodiment, a stirrer mechanism is configured to be connected to the refill capsule (H/14/24) so that a solution of skin pieces and saline is continuously stirred to maintain a uniformly suspended solution or skin pieces and liquid. Stirring is essential since two media are used (skin pieces and saline). In order to the sprayed content to be homogenous, a stirring mechanism is required. This stirring also ensures that a uniform spray of skin pieces is achieved and that it does not settle or sediment. This stirrer mechanism is a continuous stirring mechanism.

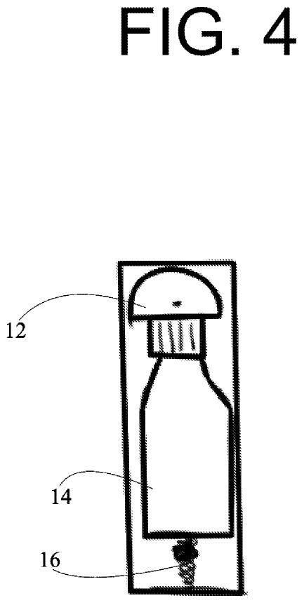

FIG. 4 illustrates an electronic stirrer mechanism of the spray device.

In at least an embodiment of the stirrer mechanism, there is provided a motorized shaker which is the electronic stirrer mechanism. This is an electronically operated vibration mechanism. It comprises a motor (12) with cam shaft which provides vibrations and, therefore, stirring action to the solution of skin pieces along with saline in a container (refill capsule) (14). This container is located on a resilient means (16) to allow for free stirring action of the container (refill capsule) (14)

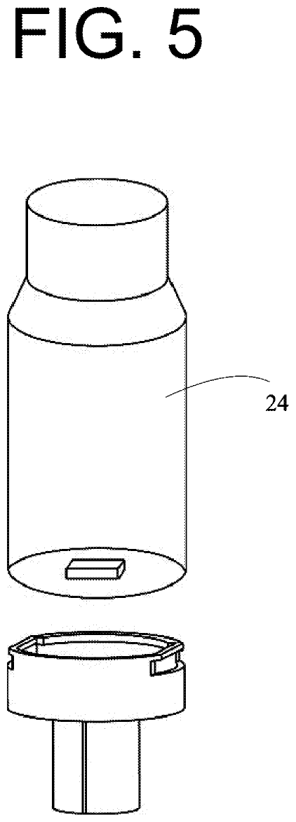

FIG. 5 illustrates a magnetic stirrer mechanism of the spray device.

In at least an embodiment of the stirrer mechanism, there is provided a magnetic shaker which comprises a container (refill capsule) (24) with a solution of skin pieces along with saline; which solution is to be shaken/stirred. There is an outer, angularly displaceable (by a motor), magnetic disc driven by a motor which cooperates magnetically with an inner magnet located inside the container to cause the container to angularly displace so as to shake/stir the contents within.

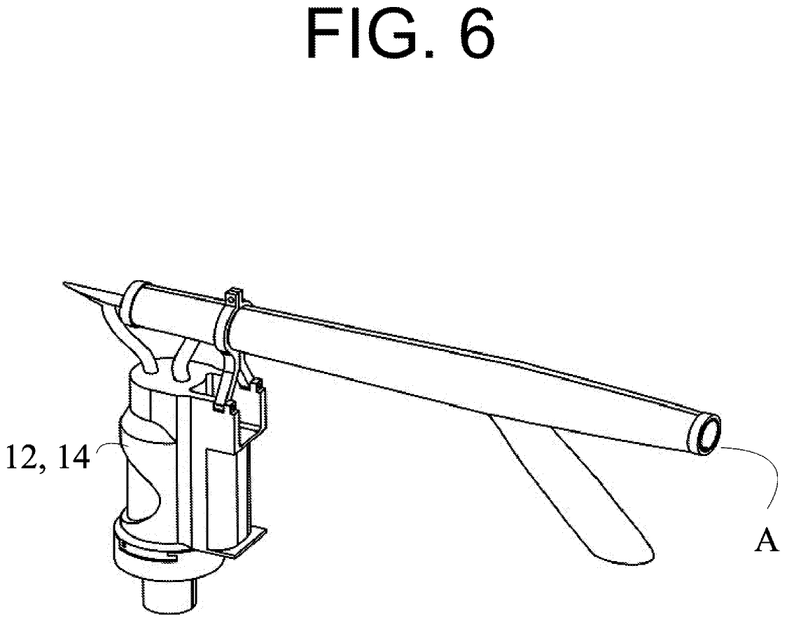

FIG. 6 illustrates a perspective view of the spray device of FIG. 2.

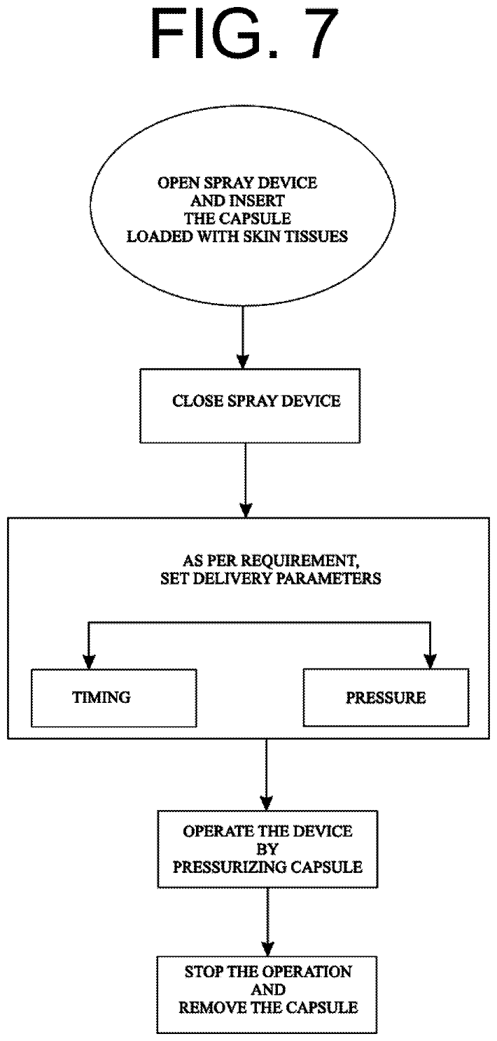

FIG. 7 illustrates a flowchart of working of this spray device.

In at least an embodiment, a nozzle attachment (A) is provided in order to spray the uniformly suspended solution from the refill capsule (H) onto a portion of the body where grafting is required. A delivery pipe feeds the solution to the nozzle (A). A main connector (C) is provided in order to detach the sample loaded (B) from the device body for refilling the capsule (H), after use. Additionally, the length of delivery pipe follows golden ration of length vis-a-vis diameter of pipe to ensure effective spray from the nozzle.

In at least an embodiment, a trigger (D) or an actuator is provided in order to trigger or enable spraying of the uniformly suspended solution on to the portion of the body where grafting is required. Compressed air is stored inside in an air container (G). The air container comprises a pressure gauge and allows a user to determine pressure of spray. A hand pump (F) is attached for manual filling of air inside the air container (G). The compressed air is released in a controlled manner with the uniformly suspended solution in order to enable effective uniformly controlled distribution of the skin cells. Air container in the electromechanical spraying system has been used as a pneumatic source which can sustain pressurized air pressure up to 5 bar. It has provision of hand pump or compressor which has been used to fill pressurized air and also pressure gauge has been used to monitor pressure in air container.

In at least an embodiment, electronics systems (E) are placed in the device to control the triggering operation with the help of the trigger (D).

Electronically controlled valves and stirrer may be provided to provide specificity and sensitivity to the device.

A flow control valve, controlled by a stepper motor, is provided to control spray through the nozzle. Stepper motor is used to vary the opening of flow control valve to control air flow emerging from the device.

According to a non-limiting exemplary embodiment, the device of this invention was tested to:
a) To find air pressure and fluid pressure at wound surface;
b) To identify optimum distance between wound and nozzle end of skin spray device;
c) To identify area covered by spray at different lengths.

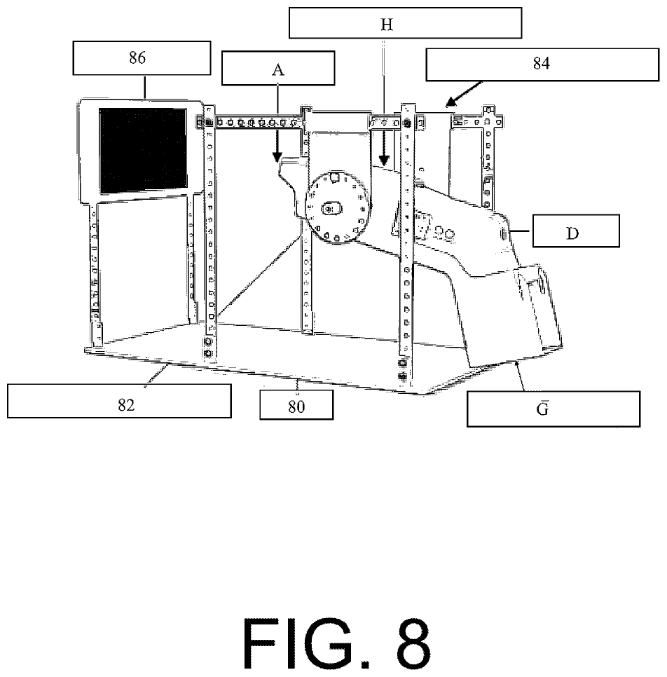

For this, a setup according to FIG. 8 was done in which:
I. A spray device, of this invention, was mounted on an angular indexing mechanism (82);
II. The minced skin tissues were loaded into the skin spray device;
III. The compressed air (G) input was provided at one end of device;
IV. The linear movement of spray device was controlled by a linear actuator (84);
V. A pressure sensor has kept in front of spray nozzle (A)—the pressure sensor (86) was considered to be equivalent to a wound surface;
VI. Distance between spray nozzle (A) tip and pressure sensor (86) has varied by using linear actuator and the displacement has measured by LVDT sensor;
VII. Air pressure and fluid pressure were measured by the pressure sensor (86);
VIII. Displacement data was measured with respect to fluid flow parameters and hence optimum distance was fixed.

Table 1, below, shows variation of pressure on wound with respect to angular position of skin spray device of this invention:

TABLE 1

| | | | Distance between | Angular | Pressure |
|---|---|---|---|---|---|
| Sr | | pressure | pressure sensor and | position | sensor reading |
| no | Input | (KPa) | spray head(mm) | (degrees) | (KPa) |
| 1 | 30 | 1.325 | 100 | 0 | 102.258 |
| | | | | 45 | 102.231 |
| | | | | 90 | 102.204 |
| | | | | 135 | 102.164 |
| | | | | 180 | 102.125 |
| 2 | 30 | 1.325 | 150 | 0 | 101.858 |
| | | | | 45 | 101.844 |
| | | | | 90 | 101.824 |
| | | | | 135 | 101.804 |
| | | | | 180 | 101.791 |
| 3 | 30 | 1.325 | 200 | 0 | 101.525 |
| | | | | 45 | 101.511 |
| | | | | 90 | 101.498 |
| | | | | 135 | 101.485 |
| | | | | 180 | 101.458 |

From Table 1, it was observed that, there is negligible variation of pressure with respect to angular position of skin spray device.

Table 2, below, shows variation of pressure with varying axial distance from wound:

TABLE 2

| | Input | Distance between | Pressure |
|---|---|---|---|
| Sr | pressure | pressure sensor and | sensor reading |
| no | (KPa) | skin spray device (mm) | (bar) |
| 1 | 301.325 | 100 | 102.204 |
| 2 | 301.325 | 110 | 102.125 |
| 3 | 301.325 | 120 | 102.018 |
| 4 | 301.325 | 130 | 101.964 |
| 5 | 301.325 | 140 | 101.884 |
| 6 | 301.325 | 150 | 101.824 |
| 7 | 301.325 | 160 | 101.738 |
| 8 | 301.325 | 170 | 101.684 |
| 9 | 301.325 | 180 | 101.618 |
| 10 | 301.325 | 190 | 101.564 |
| 11 | 301.325 | 200 | 101.498 |

Table 3, below, shows relation between distance of nozzle and pressure sensor of the skin spray device of this invention:

TABLE 3

| Sr | Distance from nozzle and | Area covered by |
|---|---|---|
| no | pressure sensor (mm) | spray (mm\hat{ }2) |
| 1 | 100 | 20 |
| 2 | 110 | 23.6 |
| 3 | 120 | 28.32 |
| 4 | 130 | 34 |
| 5 | 140 | 40.86 |
| 6 | 150 | 44.8 |
| 7 | 160 | 49 |
| 8 | 170 | 53.8 |
| 9 | 180 | 59 |
| 10 | 190 | 64.6 |
| 11 | 200 | 70.8 |

The TECHNICAL ADVANCEMENT of this invention lies in providing a spray device to spray minced skin pieces in controlled manner. Skin spray device, of this invention, is helpful in skin grafting technique for uniform distribution of skin pieces in a controlled manner. It is simple, rapid, and cost effective technique for grafting of skin over large areas with limited donor sites, and in particular to a device for spraying tiny skin pieces and distributing that on a wound surface. It has ease of application as compared to other technique used for skin grafting. The present invention provides a device with controlled methods to distribute a small area of skin over a much larger raw area, and help in faster wound healing and reducing mortality and morbidity of burn patients. This technique seeks to come up with innovative, rapid and cost effective solution for surgical management of major burns.

Advantages:
Distribution a small area of skin graft over a much larger wound area, in the ratio of more than 1:4
Stirrer system is used so that skin pieces do not settle at the bottom of capsule due to its weight.
Rapid and cost effective solution for surgical management of major burns.
Easeness by reducing the surgery timing
Comfort to doctor or surgeon because of mechanizing the manual process
Fast recovery of burn patients

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

1. A dermo-epidermal suspension spray device, the device comprising:
a refill capsule configured to contain skin pieces, wherein the refill capsule is inside a sample loader;
a stirrer configured to be connected to the refill capsule so that a solution of skin pieces and saline is continuously stirred to maintain a uniformly suspended solution of skin pieces and saline in a non-homogenous stirred manner;
a nozzle, co-axial to the refill capsule and operatively in front of the refill capsule, wherein the nozzle is configured to spray the uniformly suspended solution from the refill capsule onto a portion of a body where grafting is required;
an actuator configured to drive the uniformly suspended solution from the refill capsule through the nozzle on to the portion of body; and
an air container configured to store compressed air, wherein the air container is connected to the refill capsule so that the compressed air is used for spraying the uniformly suspended solution of skin pieces and saline through the nozzle.

2. The device as claimed in claim 1, wherein the stirrer is a motorized shaker.

3. The device as claimed in claim 1, wherein the stirrer is a motorized shaker including,
a motor with cam shaft configured to vibrate so as to stir the solution of skin pieces with saline in the refill capsule, and
a container on a resilient support to allow for free stirring action of the refill capsule.

4. The device as claimed in claim 1 wherein, the stirrer is a magnetic shaker including,
a refill capsule with a solution of skin pieces with saline to be shaken and/or stirred,
an outer, angularly displaceable, magnetic disc,
a motor configured to drive the disc, and
an inner magnet inside the refill capsule configured to cooperate magnetically with the disc and the motor to cause the refill capsule to angularly displace so as to shake and/or stir contents of the refill capsule.

5. The device as claimed in claim 1 further comprising:
a delivery pipe configured to feed the uniformly suspended solution to the nozzle, wherein the delivery pipe follows the golden ratio of length-to-diameter of pipe to ensure effective spray from the nozzle.

6. The device as claimed in claim 1 further comprising:
a main connector configured to detach a sample loaded from the device body for refilling the capsule after use.

7. The device as claimed in claim 1 wherein, the air container includes a pressure gauge displaying pressure of the spraying.

8. The device as claimed in claim 1 wherein, the air container is coupled with a hand pump attached for manual filling of air inside the air container.

9. The device as claimed in claim 1, further comprising:
electronically controlled valves in the stirrer, wherein the valves are configured to provide specificity and sensitivity to the device.

10. The device as claimed in claim 1, further comprising:
a flow control valve; and
a stepper motor controlling the flow control valve to control spray through the nozzle, wherein the stepper motor selectively opens the flow control valve to control air flow emerging from the device.
